(19) United States
(12) Patent Application Publication
(10) Pub. No.: US 2005/0192578 A1
(43) Pub. Date: Sep. 1, 2005

(54) Bone plates with locking apertures

(76) Inventor: Steven P. Horst, Dayton, OR (US)

Correspondence Address: KOLISCH HARTWELL, P.C., 520 S.W. YAMHILL STREET, SUITE 200, PORTLAND, OR, 97204, US

(21) Appl. No.: 11/071,050

(22) Filed: Feb. 28, 2005

Related U.S. Application Data
(60) Provisional application No. 60/548,685, filed on Feb. 26, 2004.

(51) Int. Cl. A61B 17/58

(52) U.S. Cl. 606/69; 606/73

(57) ABSTRACT

Systems for fixing bones, including, among others, bone plates with locking apertures, fasteners for use with the locking apertures, and methods of using the bone plates and/or fasteners for fixing bones.

### CROSS-REFERENCE TO PRIORITY APPLICATION

This application is based upon and claims the benefit under 35 U.S.C. § 119(e) of U.S. Provisional Patent Application Ser. No. 60/548,685, filed Feb. 26, 2004, which is incorporated herein by reference in its entirety for all purposes.

### BACKGROUND

The human skeleton is composed of 206 individual bones that perform a variety of important functions, including support, movement, protection, storage of minerals, and formation of blood cells. To ensure that the skeleton retains its ability to perform these functions, and to reduce pain and disfigurement, bones that are fractured or otherwise compromised should be repaired promptly and properly. Typically, such a bone is treated using one or more fixation devices, which reinforce the bone and keep it aligned during healing. Fixation devices may take a variety of forms, including external fixation devices (such as casts and fixators) and/or internal fixation devices (such as bone plates, bone screws, and nails), among others.

Bone plates are sturdy internal devices, usually made of metal, that mount directly to a bone, or bones, adjacent a fracture Or other discontinuity. To use a bone plate to repair a discontinuity of a bone, a surgeon typically (1) selects an appropriate plate, (2) reduces the discontinuity (e.g., sets the fracture), and (3) fastens the plate to bone fragments disposed on opposite sides of the discontinuity using suitable fasteners, such as screws and/or wires, so that the bone plate spans the discontinuity and fixes the bone fragments in position.

Bone plates typically include a plurality of apertures for receiving bone screws (or other fasteners). These apertures each independently may be locking or nonlocking, i.e., configured or not configured to lock a bone screw into place, respectively. Locking apertures couple a bone screw directly to a bone plate, by engaging the bone screw (e.g., by threadable engagement), thereby restricting motion of the bone screw in both axial directions. In contrast, nonlocking apertures do not couple a bone screw directly to a bone plate. Instead, nonlocking apertures rely on the ability of bone (rather than a bone plate) to grip the bone screw to hold the bone screw in engagement with the plate (and the plate in engagement with the bone). Specifically, a bone screw inserted through a nonlocking aperture will thread only into the bone (and not into the bone plate). Thus, the head of the screw can advance without limitation until it both engages the bone plate and pulls the bone plate into engagement with the bone.

Locking apertures may have a number of advantages over nonlocking apertures. First, locking apertures may be used to hold a bone plate at a fixed spacing from the bone. Such a fixed spacing may facilitate healing by (1) increasing blood flow to the bone, (2) reducing damage to the periosteum, (3) improving callous formation, (4) allowing a small, but desirable, increase in the flexibility of the fixed bone, and/or (5) reducing undesirable bonding of the plate to the bone (which is particularly important in the case of a removable bone plate). Second, locking apertures may reduce the tendency of bone screws to loosen and/or back out, and of bone plates to slip. Specifically, because nonlocking apertures do not lock the bone screw to the bone plate, only the bone prevents reverse axial movement of the bone screw. Over time, the bone screw may lose its grip on bone, particularly bone of poor quality. Accordingly, the bone screw may become loose and back out of its fully seated position, to protrude above the bone plate. Such a protruding bone screw may cause loss of fixation and/or substantial tissue irritation. Third, locking screws may relatedly reduce dependence on bone quality for fixation.

Unfortunately, despite these advantages, locking apertures may create other problems. For example, locking apertures may include a female thread configured to receive a complementary male thread formed on a proximal shaft region disposed adjacent the head of the bone screw. These female and male threads may be machine threads with a small pitch to provide substantial engagement of these threads and thus restrict undesired reverse axial movement of the bone screw. However, the distal shaft region of the bone screw may have a different thread with a larger pitch to facilitate more rapid advancement of the bone screw into bone. Accordingly, the bone screw may have a pitch differential so that the bone screw, when engaged with the locking aperture, advances farther, per revolution, in bone than within the locking aperture, to compress the bone plate against the bone. In some cases, this compression may be desirable or may be accommodated by spacing the bone plate from the bone when the bone screw is first placed into bone. However, once the bone plate is compressed against bone by placement of a first bone screw fully into a locking aperture, further compression at other adjacent locking apertures may not be desirable. For example, threads cut into bone by screws placed at these other locking apertures (or the threads of the other locking apertures) may be stripped as these additional bone screws are locked to the bone plates.

Locking apertures also may provide less flexibility than nonlocking apertures for placement of bone screws. In particular, locking apertures generally are configured as circular openings that include a helical rib on the wall of the opening. Such circular openings may define a single position at which a bone screw may be placed into bone. In contrast, nonlocking openings may be configured as elongate openings or slots that permit a surgeon greater freedom in selecting a suitable position for screw placement from a range of permitted positions along the elongate openings. However, because they are elongate, slots cannot be configured to be nonlocking with a helical rib that extends along their length.

### SUMMARY

The present teachings provide systems for fixing bones, including, among others, bone plates with locking apertures, fasteners for use with the locking apertures, and methods of using the bone plates and/or fasteners for fixing bones.

### BRIEF DESCRIPTION OF THE DRAWINGS

FIG. 1 is a sectional view of a region of an exemplary bone plate with a locking aperture, shown with an exemplary bone fastener retained in the locking aperture, in accordance with aspects of the present teachings.

FIG. 2 is a fragmentary, top plan view of the locking aperture of FIG. 1, viewed generally along line 2-2 of FIG. 1, in the absence of the bone fastener.

FIG. 3 is a fragmentary, top plan view of a combination aperture having adjacent locking and nonlocking regions, in accordance with aspects of the present teachings.

FIG. 4 is a first sectional view of the combination aperture of FIG. 3, taken generally along line 4-4 of FIG. 3, with an exemplary bone screw retained in the locking region of the combination aperture, in accordance with aspects of the present teachings.

FIG. 5 is a second sectional view of the combination aperture of FIG. 3, taken generally along line 4-4 of FIG. 3, with another exemplary bone screw retained in the locking region of the combination aperture, in accordance with aspects of the present teachings.

FIG. 6 is a sectional view of a region of a bone plate with another exemplary locking aperture, shown With an exemplary bone fastener retained in the locking aperture, in accordance with aspects of the present teachings.

FIG. 7 is a sectional view of a region of a bone plate with yet another exemplary locking aperture, shown with an exemplary bone fastener retained in the locking aperture, in accordance with aspects of the present teachings.

FIG. 8 is a somewhat schematic, sectional view of a bone plate secured to a region of a bone using a bone screw received in the bone through a locking aperture of the bone plate, in accordance aspects of with the present teachings.

FIG. 9 is a somewhat schematic, sectional view of a bone plate secured to a region of a bone using a bone screw received distally in a locking aperture of the bone plate after passing through the bone, in accordance with aspects of the present teachings.

FIG. 10 is a somewhat schematic, sectional view of two bone plates or plate portions secured to a region of a bone using a bone screw passing through bone from a first of the plates or plate portions and into engagement with a locking aperture of a second of the plates or plate portions, in accordance with aspects of the present teachings.

### DETAILED DESCRIPTION

The present teachings provide systems for fixing bone, including, among others, bone plates with locking apertures or combination locking/nonlocking apertures, fasteners for use with the locking or combination locking/nonlocking apertures, and methods of the using the bone plates and/or fasteners for fixing bones. The bone plates may include a wall defining an aperture configured to allow coupling of a fastener to the plate. The wall may include a pair of opposing retention structures, such as ridges, recesses, and/or detents, disposed on opposing sides of the aperture. The retention structures may be linear, circular, and/or angular, among others.

In some embodiments, the retention structures may be offset longitudinally from one another; that is, offset in a direction at least generally perpendicular to a plane defined by the aperture, and/or at least generally parallel to a long axis of a fastener configured to be received by the aperture, so that one of the retention structures is closer to a bone-facing surface of the bone plate (and thus to a surface of bone) than another of the retention structures. In some examples, the retention structures may be offset longitudinally by a distance related to the pitch of a threaded shank of a fastener to be received in the aperture. In some examples, a portion of the aperture may be widened and/or may lack the retention structures, to provide a combination locking/nonlocking aperture. Overall, the systems for bone fixation provided herein may combine the flexibility of fastener placement offered by nonlocking apertures, with the stability and control offered by locking apertures.

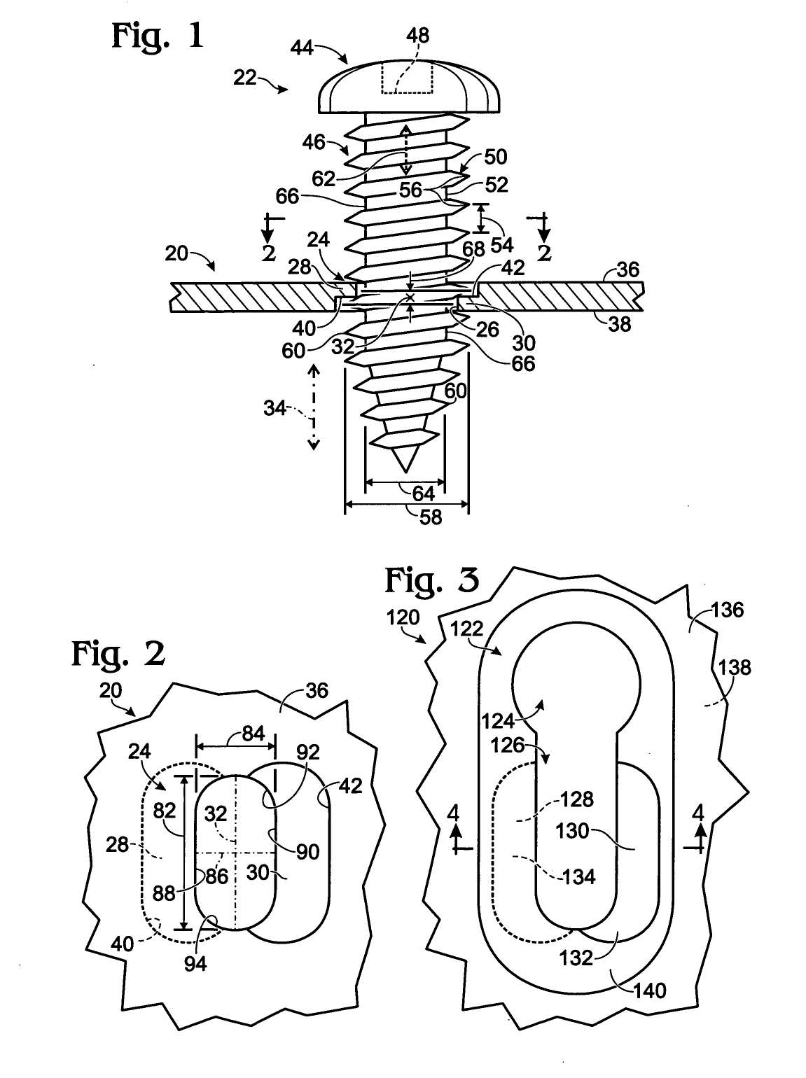

FIG. 1 shows a sectional view of a region of an exemplary bone plate 20 retaining a bone fastener 22 in a locking aperture 24 of the bone plate. Locking aperture 24 may be configured so that bone fastener 22 can be advanced into the aperture by rotation, to be retained therein by engagement of threads of the fastener with retention structures flanking the aperture, as described below.

Locking aperture 24 may be defined by a wall 26 formed by the bone plate. Wall 26 may include opposing retention structures, such as ledges or ridges 28, 30, disposed at least generally opposite one another across a long axis 32 of the aperture. Ridges 28, 30 may be offset from each other longitudinally; that is, offset parallel to an axis 34 extending at least generally orthogonally through the plate and at least generally parallel to a longitudinal axis of a fastener to be inserted into the plate. Ridges 28, 30 thus may be disposed at different positions relative to an outer surface 36 and an inner surface 38 of the plate. For example, first ridge 28 may be spaced from inner surface 38 by an inner recess 40 formed in the inner surface. Alternatively, or in addition, second ridge 30 may be spaced from outer surface 36 by an outer recess 42 formed in the outer surface 36.

Bone fastener 22 may include a head 44 and a threaded shank 46. The head may provide tool engagement structure 48 to permit the fastener to be rotated and thus advanced into bone and through the aperture. Engagement structure 48 may be, for example, one or more notches to receive a screwdriver tip, a recess to receive an allen wrench, or any other structure suitable for receiving a tool configured to rotate the fastener. The bone fastener may be oriented as shown in FIG. 1, or disposed in an inverted orientation relative to the inner and outer surfaces of the bone plate, to define the direction of travel of the fastener to/from the plate. The threaded shank may include one or more threads 50 formed on a core or shaft 52 of the shank. Each thread may be configured as a helical rib (or spaced sections thereof) having a pitch 54 measured between adjacent thread segments 56 of the helical rib. The threaded shank may have a major diameter 58 measured from crest to crest 60 of the thread across the central axis 62 of the shank. The threaded shank also may have a minor diameter 64 measured from root to root 66 of the thread across the central axis, or equivalently measured as the diameter of core 52. A fastener may be selected so that its major diameter is greater than the lateral spacing of opposing ridges 28, 30 (generally, the width 84 of the aperture; see FIG. 2). The fastener also may be selected so that its minor diameter is less than the lateral spacing of the opposing ridges 28, 30, or so that its minor diameter is approximately equal to this or even somewhat greater than this lateral spacing (for example, to provide an interference fit). In some embodiments, the fastener may be selected so that pitch 54 is approximately twice the longitudinal offset 68 of opposing ridges 28, 30.

FIG. 2 shows locking aperture 24 of bone plate 20, viewed from above outer surface 36 of the bone plate. In some embodiments, locking aperture 24 may be at least generally circular. Alternatively, as FIG. 2 depicts, the locking aperture may be elongate, having a length 82, measured parallel to long axis 32, that is greater than its width 84, measured parallel to transverse axis 86. In some embodiments, the locking aperture may be at least generally oval-shaped, having linear sides 88, 90 along which opposing ridges 28, 30 at least partially extend. Accordingly, opposing ridges 28, 30, or portions thereof, may be at least substantially parallel to long axis 32. Alternatively, the sides of the locking aperture may be nonlinear, for example, curved (arcuate) or angular, among others. Fastener 22 (see FIG. 1) may be coupled to the bone plate at any desired position selected from a range of possible positions disposed along long axis 32. For example, inner recess 40 (show in dashed outline) and outer recess 42 may extend partially or completely along linear sides 88, 90 of locking aperture 24 to define ridges 28, 30. These recesses and/or the ridges also may extend any suitable distance into circular end regions 92, 94 of the aperture. Thus, locking aperture 24 may combine the advantages of nonlocking and locking apertures, by allowing flexible location of the fastener, while still providing locking engagement of the fastener to the bone plate.

Further aspects of the present teachings are described in the following sections, including (I) general aspects of bone plates with locking apertures, (II) locking apertures, (III) fasteners for locking apertures, and (IV) examples.

### I. GENERAL ASPECTS OF BONE PLATES WITH LOCKING APERTURES

Bone plates as described herein generally comprise any relatively low-profile (or plate-like) fixation device configured to stabilize at least one bone by attachment to the bone. The fixation device may be configured to span a bone discontinuity so that the fixation device fixes the relative positions of bone portions/fragments (and/or bones) disposed on opposing sides of the bone discontinuity. Alternatively or in addition, the fixation device may provide structural support to a bone either having or lacking a discontinuity.

Discontinuities treatable with bone plates may occur naturally or may result from injury, disease, and/or surgical intervention, among others. Accordingly, exemplary discontinuities for use with the bone plates described herein may include joints, fractures (breaks in bones), osteotomies (cuts in bones), and/or nonunions (for example, produced by injury, disease, or a birth defect), among others.

The bone plates described herein may be configured for use on any suitable bone of the human skeleton and/or of the skeleton of another vertebrate species. Exemplary bones may include bones of the arms (radius, ulna, humerus), legs (femur, tibia, fibula, patella), hands/wrists (e.g., phalanges, metacarpals, and carpals), feet/ankles (e.g., phalanges, metatarsals, and tarsals), vertebrae, scapulas, pelvic bones, cranial bones, ribs, and and/or clavicles, among others. Particular examples where bone plates having locking apertures may be suitable include, but are not limited to, discontinuities in adjacent metaphyseal and/or diaphyseal regions of long bones, such as proximal or distal regions of the humerus, tibia, femur, rib bones, and/or clavicle.

Each bone plate may be configured to be disposed in any suitable position relative to its target bone. The bone plate (or a plate portion) may be configured to be disposed in contact with an exterior surface of the bone, and thus may be positioned substantially (or completely) exterior to the bone. Alternatively, the bone plate may be configured to be disposed at least partially interior to a bone, that is, apposed to (normally) interior bone surfaces when secured to the bone. The interior bone surfaces may be accessed surgically during installation of the bone plate (such as by punching the bone plate through the exterior bone surface) and/or may be accessible due to a break, a cut, or the like.

The bone plates of the present teachings may be formed of any suitable materials. The plates may be of a sturdy yet malleable construction. Generally, the bone plates should be at least as stiff and strong as the section of bone spanned by the plates, yet flexible (e.g., springy) enough so as not to strain the bone significantly. Suitable materials for forming the bone plates may include biocompatible materials (such as titanium or titanium alloys, cobalt chromium, stainless steel, plastic, ceramic, etc.) and/or bioabsorbable materials (such as polygalactic acid (PGA), polylactic acid (PLA), copolymers thereof, etc.), among others.

The bone plates may be configured to reduce irritation to a treated bone and surrounding tissue. For example, the plates may be formed of a biocompatible material, as described above. In addition, the bone plates may have a low and/or feathered profile to reduce their protrusion into adjacent tissue, and rounded, burr-free surfaces to reduce the effects of such protrusion.

The bone plates described herein may be sized and shaped to conform to particular bones, or to particular portions of a bone (or bones). The plates may be generally elongate, with a length L, a width W, and a thickness T. Here, length L≧width W≧thickness T. In use, the long axis of the bone plates (or of a plate portion) may be aligned With the long axis of the corresponding bone, or may extend obliquely or transversely relative to the bone's long axis. The length and/or width of the bone plates may be varied according to the intended use, for example, to match a plate's shape with a preselected region of bone(s) and/or with a particular injury to the bone. For example, the plates may be generally linear for use on the shaft of a long bone, or may have a nonlinear shape for use near an end of a bone and/or for transverse placement on the shaft, among others. In some examples, the plates may be configured to wrap at least partially around a bone, so that portions of each plate are disposed on generally opposing sides/surfaces of a bone. The generally opposing sides/surfaces may have relative positions that are anterior and posterior, medial and lateral, superior and inferior, proximal and distal, or the like. In some embodiments, the bone plates may be configured for use on both sides of the body, such in which case the plates may be bilaterally symmetrical. In some embodiments, the bone plates may be asymmetrical and specifically configured for use on one Or the other of either the left or the right side of the body.

The bone plates may include inner (bone-facing) and outer (bone-opposing) surfaces. One or both of these surfaces may be contoured generally to follow an exterior surface of a target bone (or bones) for which a bone plate is intended, so that the bone plate maintains a low profile and fits onto the bone(s) in an appropriate manner. For example, the inner surface of a plate (or of an exterior plate portion) may be generally complementary in contour to the bone surface. The outer surface of the plate (or of an exterior plate portion) also may correspond in contour to the bone surface and may be generally complementary to the inner surface of the plate. The bone plates may be partially Or completely precontoured at the time of manufacture, allowing practitioners to apply the plates to one or more bones with little or no additional bending at the time of application. Alternatively or in addition, the bone plates may be custom-contoured by practitioners before and/or during installation onto bone.

The thickness of the bone plates may be defined by the distance between the inner and outer surfaces of the plates. The thickness of the plates may vary between plates and/or within the plates, according to the intended use. For example, thinner plates may be configured for use on a smaller bone and/or on a bone or bone region where soft tissue irritation is a greater concern. Thickness also may be varied within the plates. For example, the plates may become thinner as they extend over protrusions (such as processes, condyles, tuberosities, and/or the like), reducing the profile and/or rigidity of the plate in those regions, among others. Alternatively or in addition, the thickness may vary in regions where an interior portion of the bone plate extends into bone. For example, in those regions, the plate may become thinner to facilitate insertion of this interior portion, or thicker to increase structural stability. The thickness of the plates also may be varied to facilitate use. For example, plates may be thinner in regions where they typically need to be deformed by bending and/or twisting the plates, such as at a junction (or bridge region) between plate portions. On the other hand, the plates may be thicker (and thus typically stronger) in regions where they may not need to be contoured, such as along the shaft of a bone. In some embodiments, the thickness may decrease selectively in regions adjacent openings or apertures of the bone plates, to form retention structures of locking apertures.

Bone plates according to the present teachings generally include a plurality of openings or apertures. The openings may be adapted to receive fasteners for securing the plates to bone. Alternatively or in addition, the openings may be adapted to alter the local rigidity of the plates, to permit the plates to be manipulated with a tool (such as an attachable handle), to facilitate blood flow to bone regions where the bone plates are installed, to promote healing, and/or the like.

The openings may have any suitable positions, sizes, and/or densities within each portion of a bone plate. The openings may be arrayed generally in a line along a portion of the plate; for example, the openings may be centered across the width of the plate. Alternatively, the openings may be arranged nonlinearly, such as disposed in an arcuate, staggered or other two-dimensional (or three-dimensional) arrangement. In some embodiments, the bone plates may include at least one pair of openings that are aligned, so that a fastener can extend concurrently into each opening of the pair. One or both of the openings of the aligned pair may be a locking aperture. In some embodiments, one of the openings may be an elongate locking aperture and the other of the openings may be a substantially corresponding and aligned elongate aperture that is nonlocking.

In some embodiments, the openings may be configured so that a set of bone screws can be directed along nonparallel paths, which may increase the purchase of the set of bone screws on bone. Further aspects of openings configured to direct bone screws, particularly unicortical bone screws, along nonparallel paths are included in the following patent application, which is incorporated herein by reference: U.S. patent application Ser. No. 10/968,850, filed Oct. 18, 2004.

The openings may have any suitable shape and structure. Exemplary shapes include circular, elongate (such as elliptical, rectangular, oval), etc. The openings also may include counterbores, which may be configured, for example, to receive a head of a bone screw, to reduce or eliminate protrusion of the head above the outer surface of the plate. The openings may be threaded or unthreaded, and each bone plate may include one or more threaded and/or unthreaded openings. In some embodiments, the plates may include one or a plurality of elongate openings (slots) extending axially, obliquely, and/or transversely within each bone plate. The elongate openings May be, for example, compression slots that include tapered counterbores to provide compression when heads of bone screws are advanced against the counterbores. Alternatively, or in addition, the elongate openings may be used to adjust the position of bone plates and/or plate portions relative to bone before the plates are fully secured to the bone. One or more of the elongate openings of each bone plate may be a locking aperture (see Section II below).

In some embodiments, the bone plates may include one or more projections that extend generally orthogonally from an inner surface of the bone plates toward bone. The projections may be sharp or blunt according to their intended use. For example, sharp projections may be configured as prongs that penetrate bone to restrict movement of the bone plates. Prongs may be used in place of, or in addition to, bone fasteners, for one or more portions of each bone plate. Blunt projections, such as ridges or knobs, may be configured as spacing members that elevate the bone plates from the bone surface. Locking apertures may facilitate holding bone plates at such elevated or spaced positions from bone.

The bone plates may have at least one, and generally two or more, plate portions (or anchor portions) configured to be secured to different regions of a bone (or bones). Each plate portion may be structured for a specific region of a bone. For example, the bone plates may include a proximal plate portion for attachment to a more proximal region of a bone, and a distal plate portion for attachment to a more distal region of the same bone. Alternatively or in addition, the bone plates may include an exterior plate portion configured to fit against an exterior surface region of bone adjacent a bone discontinuity, and/or an interior plate portion configured to be received in an interior (e.g., recessed, resected, and/or excavated) region of bone adjacent the bone discontinuity.

The plate portions of a bone plate may have any suitable connection. In some embodiments, two or more of the plate portions (or the entire bone plate) may be formed integrally, so that one contiguous piece of the bone plate includes those plate portions. Alternatively, plate portions may be formed as separate pieces. The separate pieces may be connected by any suitable connection and/or joint, including one or more fasteners, welding, a hinge joint, a ball in socket, and/or the like. Further aspects of bone plates having adjustable joints are described in the following patent application, which is incorporated herein by reference: U.S. patent application Ser. No. 10/716,719, filed Nov. 19, 2003. In some embodiments, the bone plates may be two separate bone plates configured to be connected by one or more fasteners, such as fasteners that extend from one of the bone plates, through bone, and into engagement with another of the bone plates.

In some embodiments, a single bone plate may include plate portions configured to be spanned by a fastener extending through bone between the plate portions. The plate portions of a bone plate may have any suitable relative disposition. In some embodiments, the plate portions may define planes that are parallel and offset from each other along an axis extending at least generally orthogonal to the planes. In some embodiments, the plate portions may define planes that are disposed oblique or at least substantially transverse to each other. For example, the bone plate may have an exterior plate portion that fits against an exterior surface of a bone, and an interior plate portion that extends at least substantially transverse to the exterior surface to be received in the interior of the bone. The relative disposition of the plate portions may be fixed and/or adjustable. In some embodiments, the plate portions may be connected integrally by a deformable bridge region, so that the bone plate can be bent pre-operatively or peri-operatively to adjust the relative disposition of the plate portions. Alternatively, the plate portions may be distinct pieces connected, for example, through an adjustable joint.

Each plate portion may have one or more openings or apertures, and each opening may be configured to receive a bone fastener for placement of the bone fastener into bone. Alternatively or in addition, an opening may be configured to enable the fastener to extend between an opening in each of two or more plate portions, such as between a first opening in a first plate portion, and a locking aperture in a second plate portion.

The first opening in the first plate portion may have any suitable configuration. For example, the first opening may be locking or nonlocking, and may be configured to be disposed adjacent a head of a fastener. In this case, the locking aperture to which the fastener extends from the first opening may be configured to be disposed adjacent a distal region of a shank of the fastener. Alternatively, the first opening may be configured to be disposed adjacent a distal region of the shank of the fastener, and the locking aperture may be configured to be disposed adjacent a head of a fastener. The first opening may be threaded or unthreaded, and may be circular or elongate. Accordingly, the first opening may restrict a fastener of a suitable diameter to substantially one direction of approach (such as with a circular first opening and a close-fitting fastener), or the first opening may be an elongate opening that permits a fastener be placed at a range of positions along the opening. In some embodiments, the first opening may be elongate and may define an axis (the long axis of the opening) that is at least substantially parallel with an axis defined by the locking aperture (the long axis of the locking aperture). Accordingly, the first opening and the locking aperture may be disposed so that the fastener can be directed from the first opening to a plurality of positions disposed along the long axis of the locking aperture. In some embodiments, the first plate portion may include two or more first openings that permit two or more fasteners to be received and retained, alternately and/or concurrently, in the locking aperture.

An interior plate portion may be configured for installation into bone. Accordingly, the interior plate portion may be thinner than the exterior plate portion. Alternatively or in addition, the interior plate portion may have tapered edges, particularly a leading edge that enters bone first, so that the leading edge can penetrate bone more easily. The interior plate portion may have fewer openings than the exterior plate portion. In some embodiments, the interior plate portion may have one or more locking apertures and no additional openings. Alternatively, the interior plate portion may have one or more additional (nonlocking) openings.

### II. LOCKING APERTURES

Bone plates according to the present teachings may include one or more locking apertures. In some embodiments, the bone plates may include a locking aperture in each anchor portion of the bone plates. Each locking aperture may have one or more retention structures configured to engage and retain a threaded fastener. A pair of the retention structures may be disposed on opposing sides of the aperture and offset longitudinally from each other, i.e., offset from each other in a direction at least substantially orthogonal to a plane defined by the aperture. Each opposing side of the aperture may have one retention structure, or a plurality of two or more retention structures, such as two or more ridges separated longitudinally. That is, two or more retention structures on the same side of an aperture may be separated from each other in a direction orthogonal to the plane defined by the aperture.

A locking aperture may have any suitable shape and disposition. The locking aperture may be generally circular or may be elongate, such as oval shaped. The locking aperture may be centered across the width of a bone plate, or may be disposed asymmetrically with respect to the width. When elongate, the long axis of the locking aperture may extend in a direction at least substantially parallel to the long axis of the bone plate, Or obliquely or transversely to the long axis. The locking aperture may be linear or nonlinear, such as arcuate and/or angular, among others.

The retention structures may have any suitable shape, disposition, and size. For example, the retention structures may be disposed as uninterrupted ledges or ridges that each extend along a portion of a wall or perimeter of the aperture. Alternatively, each retention structure may be formed by a set of interrupted ridges (spaced ridge segments) or projections arrayed along an edge of the aperture. Each retention structure thus may define a line or an arc extending along at least a portion of one edge of the aperture. The line may be at least substantially parallel to a long axis of the aperture. An opposing retention structure disposed along an opposite edge of the aperture also may be parallel to the long axis of the aperture, and thus opposing retention structures may be disposed at least partially parallel to each other. Alternatively or in addition, the line defined by a retention structure may be at least substantially parallel to a plane defined by the aperture, or may extend obliquely thereto.

The arc defined by a retention structure may be nonhelical, that is, defining a plane. This plane may be at least substantially parallel to a length-by-width plane defined by the aperture, or may extend obliquely to the plane defined by the aperture. The retention structures may have a generally constant spacing along at least a portion of a locking aperture. The retention structures may have any cross-sectional shape including angular (such as rectilinear or forming a sharp edge), and/or rounded, among others. The thickness of each retention structure, as measured parallel to an axis extending orthogonally between the outer and inner surfaces of the plate, may be less than the pitch of a thread to be received by the locking aperture, so that the retention structure can be received between adjacent thread segments/crests of a thread of a fastener. The retention structures may be longitudinally offset by any suitable distance. This distance may be determined, in part, by the thickness of the plate and/or by the pitch of a fastener thread to be placed into the aperture.

Retention structures may be at least partially defined and positioned by recesses or detents formed in the inner and/or outer surfaces of the bone plate. The recesses may have a width that is equal to or greater than the height of a thread (measured from crest to trough) on a fastener to be used with the retention structures. The recesses may be formed by any suitable operation, including machining, molding, shaping, etc. A recess may be formed on a different surface (i.e., the interior or exterior of the plate) of each opposing side of a locking aperture, so that the recesses are longitudinally offset from each other. Alternatively, recesses may be formed on each of the two surfaces of each opposing side of a locking aperture. This configuration may be suitable, for example, in cases of thicker plates and/or fasteners with a smaller pitch. In embodiments where recesses are formed on each of the interior and exterior surfaces of the plate and on each opposing side of the locking aperture, the recesses still may be longitudinally offset from each other, for example if the recesses are formed to have different longitudinal thicknesses on the two opposing sides of the aperture.

Locking apertures may be formed in plates having any suitable thickness. For example, the thickness of the plate may relate to the offset of the retention structures of a locking aperture. In some embodiments, the thickness of the plate may be about twice the offset. Accordingly, since as previously described the thread pitch of a fastener designed for use in the aperture also may be about twice the offset distance, locking apertures configured for use with threaded fasteners may have a thickness that is approximately equal to the thread pitch of the fasteners (see Table I of Section III below for exemplary thread pitches). In some cases, locking apertures may be formed on relatively thin plates having a thickness of less than about two mm, or less than about one mm.

Locking apertures described herein may have advantages over nonlocking apertures. For example, locking apertures may allow at least a portion of the plate optionally to be positioned away from the bone. This positioning away from the bone may allow the periosteum, neurovascular bundle, and the like, to pass under the plate without being pinched or damaged, possibly promoting increased blood flow to the fractured or otherwise compromised area of bone and/or promoting faster healing of the bone. The positioning away further may allow for some amount of natural setting and/or thickening (e.g., through calcification) of a fractured or otherwise injured bone.

### III. FASTENERS FOR LOCKING APERTURES

Fasteners suitable for use with locking apertures generally comprise any mechanism for affixing a bone plate to a bone, including screws, pins, and/or wires, among others. Bone screws may include unicortical, bicortical, and/or cancellous bone screws. Unicortical and bicortical bone screws typically have relatively small threads for use in hard bone, such as is typically found in the shaft portion of a bone, whereas cancellous bone screws typically have relatively larger threads for use in soft bone, such as is typically found near the ends (metaphyseal regions) of a long bone. Unicortical bone screws penetrate the bone cortex once, adjacent the bone plate, whereas bicortical bone screws penetrate the bone cortex twice, once adjacent the bone plate and again opposite the bone plate. Generally, unicortical screws provide less support than bicortical screws, because they penetrate less cortex. The size and shape of the fasteners may be selected based on the size, shape, and retention-structure configuration of the locking apertures, or vice versa.

Each fastener placed into a locking aperture may have a threaded shank. The threaded shank may have one thread (single-threaded) or a plurality of threads (e.g., double-threaded, triple-threaded, etc.). The threads may be interspersed, so that the shank is multi-threaded, for example, to accommodate a greater pitch (a steeper thread angle). Alternatively, or in addition, the threads may be disposed on adjacent and/or nonoverlapping regions of the shank. The pitch of a thread may be constant along the shank, or may change either continuously or discretely according to position, For example, the pitch may decrease closer to a head of the fastener, to provide compression of the bone as the fastener is advanced into the bone. In some embodiments, the threaded shank may have two or more distinct threads with different pitches, such as a distal thread with a greater pitch, and a proximal thread with a lesser pitch, or vice versa. The proximal or the distal thread (or both) may be configured to be retained by a locking aperture.

In some embodiments, the threads of the threaded shank may have an at least substantially constant pitch along the shank. In these embodiments, the rate of advancement of the threaded shank into bone may be at least substantially equal to the rate of advancement of the threaded shank through the locking aperture, to restrict compression of the bone plate against the bone and to preserve any desired spacing between the plate and the bone as the fastener is fully advanced into the aperture. The pitch of a thread on a threaded shank may be selected according to the vertical offset (or the thread pitch) of a locking aperture, in some embodiments so that the pitch is about twice the vertical offset. With two interspersed threads on a shank, the longitudinal offset of the retention structures of a locking aperture, relative to one another, may be zero, i.e. the retention structures may be aligned in a plane at least substantially parallel to a plane defined by the aperture itself.

The threaded fasteners may have any suitable linear density of threads (or linear densities, if multithreaded). These densities may be measured using units, for example, such as number of threads per inch and/or meter, among others. For example, the fastener may have 16, 20, 24, 28, 32, 36, 40, and/or other numbers of threads per inch, among others; these linear densities correspond to thread-to-thread spacings (or pitches) of 0.0625 inches, 0.0500 inches, 0.0417 inches, 0.0357 inches, 0.03125 inches, 0.0278 inches, 0.0200 inches, and/or other fractions of an inch. In some embodiments, the threads on the fastener may have a continuously or discontinuously varying pitch at different positions along the fastener axis. Typically, in apertures having retention structures as described herein, the retention structures may be longitudinally offset from each other by less than (often one half of) the thread-to-thread spacing. Thus, typical offsets may include 0.03125 inches, 0.02500 inches, 0.02083 inches, 0.01786 inches, 0.01562 inches, 0.01389 inches, 0.01250 inches, and/or other numbers of inches. Table I shows additional exemplary standard and metric-based linear thread densities and corresponding thread-to-thread spacings. Table I also shows plate offsets (for retention structures) for the simple case in which the longitudinal offset of the retention structures is one-half the thread-to-thread spacing.

TABLE I

| | |
|---|---|
| Standard | Metric |

| | | Thread | Plate | Thread | Plate |
|---|---|---|---|---|---|
| Threads | | Spacing | Offset | Spacing | Offset |
| (#/Inch) | | (Inches) | (Inches) | (mm) | (mm) |
| | 2 | 0.50000 | 0.25000 | 0.10 | 0.050 |
| | 4 | 0.25000 | 0.12500 | 0.20 | 0.100 |
| | 6 | 0.16667 | 0.08333 | 0.25 | 0.125 |
| | 8 | 0.12500 | 0.06250 | 0.30 | 0.150 |
| | 10 | 0.10000 | 0.05000 | 0.40 | 0.200 |
| | 12 | 0.08333 | 0.04167 | 0.50 | 0.250 |
| | 14 | 0.07143 | 0.03571 | 0.60 | 0.300 |
| | 16 | 0.06250 | 0.03125 | 0.70 | 0.350 |
| | 18 | 0.05556 | 0.02778 | 0.75 | 0.375 |
| | 20 | 0.05000 | 0.02500 | 0.80 | 0.400 |
| | 22 | 0.04545 | 0.02273 | 0.90 | 0.450 |
| | 24 | 0.04167 | 0.02083 | 1.00 | 0.500 |
| | 26 | 0.03846 | 0.01923 | 1.10 | 0.550 |
| | 28 | 0.03571 | 0.01786 | 1.20 | 0.600 |
| | 30 | 0.03333 | 0.01667 | 1.25 | 0.625 |
| | 32 | 0.03125 | 0.01563 | 1.30 | 0.650 |
| | 34 | 0.02941 | 0.01471 | 1.40 | 0.700 |
| | 36 | 0.02778 | 0.01389 | 1.50 | 0.750 |
| | 38 | 0.02632 | 0.01316 | 1.60 | 0.800 |
| | 40 | 0.02500 | 0.01250 | 1.70 | 0.850 |
| | 42 | 0.02381 | 0.01190 | 1.75 | 0.875 |
| | 44 | 0.02273 | 0.01136 | 1.80 | 0.900 |
| | 46 | 0.02174 | 0.01087 | 1.90 | 0.950 |
| | 48 | 0.02083 | 0.01042 | 2.00 | 1.000 |
| | 50 | 0.02000 | 0.01000 | 2.10 | 1.050 |
| | 52 | 0.01923 | 0.00962 | 2.20 | 1.100 |
| | 54 | 0.01852 | 0.00926 | 2.25 | 1.125 |
| | 56 | 0.01786 | 0.00893 | 2.30 | 1.150 |
| | 58 | 0.01724 | 0.00862 | 2.40 | 1.200 |
| | 60 | 0.01667 | 0.00833 | 2.50 | 1.250 |
| | 62 | 0.01613 | 0.00806 | 2.60 | 1.300 |
| | 64 | 0.01563 | 0.00781 | 2.70 | 1.350 |
| | 66 | 0.01515 | 0.00758 | 2.75 | 1.375 |
| | 68 | 0.01471 | 0.00735 | 2.80 | 1.400 |
| | 70 | 0.01429 | 0.00714 | 2.90 | 1.450 |
| | 72 | 0.01389 | 0.00694 | 3.00 | 1.500 |
| | 74 | 0.01351 | 0.00676 | 3.10 | 1.550 |
| | 76 | 0.01316 | 0.00658 | 3.20 | 1.600 |
| | 78 | 0.01282 | 0.00641 | 3.25 | 1.625 |
| | 80 | 0.01250 | 0.00625 | 3.30 | 1.650 |
| | | | | 3.40 | 1.700 |
| | | | | 3.50 | 1.750 |
| | | | | 3.60 | 1.800 |
| | | | | 3.70 | 1.850 |
| | | | | 3.75 | 1.875 |
| | | | | 3.80 | 1.900 |
| | | | | 3.90 | 1.950 |
| | | | | 4.00 | 2.000 |

The threaded fasteners may have any suitable diameters, including major (crest-to-crest) and minor (root-to-root) diameters. In some embodiments, the major diameters may be between about 1 to 10 mm. Exemplary major diameters include 1 mm, 1.5 mm, 2.0 mm, 2.7 mm, 3.5 mm, and 4.0 mm. In some embodiments the difference between the major and minor diameters (generally, twice the thread height) may be in the range of about 0.1 mm to 5 mm, or in the range of about 0.2 mm to 2 mm. In some embodiments, the major diameter and minor diameter of the threaded shank may be generally constant along the length of the shank. In other embodiments, these diameters may be different in proximal and distal portions of the shank. For example, a proximal region of the shank (adjacent the head of the fastener) may have both a greater major and a greater minor diameter than a distal region of the shank, to permit selective coupling of the proximal region to a locking aperture. Further aspects of bone screws with varying diameter and/or pitch that may be suitable for use in locking apertures are described in the following patent application, which is incorporated herein by reference: U.S. Provisional Patent Application Ser. No. 60/480,517, filed Jun. 20, 2003.

Each fastener to be placed in a locking aperture may have a head. The head may have any suitable tool engagement structure, such as a hexagonal recess, a single slot, a pair of slots in a cruciform arrangement, etc.

### IV. EXAMPLES

The following examples describe selected aspects and embodiments of the present teachings, including exemplary bone plates with locking apertures, fasteners configured to be used with the locking apertures, and methods of using the bone plates and/or fasteners to fix bones. These examples and the various features and aspects thereof are included for illustration and are not intended to limit or define the scope of the invention.

### Example 1

### Bone Plates with Combination Locking/Nonlocking Apertures

This example describes exemplary bone plates having combination apertures with distinct regions that are locking and nonlocking; see FIGS. 3-5. In some examples, combination apertures may permit a surgeon to select compressed or noncompressed (i.e., spaced or nonspaced) engagement of a bone plate with a bone.

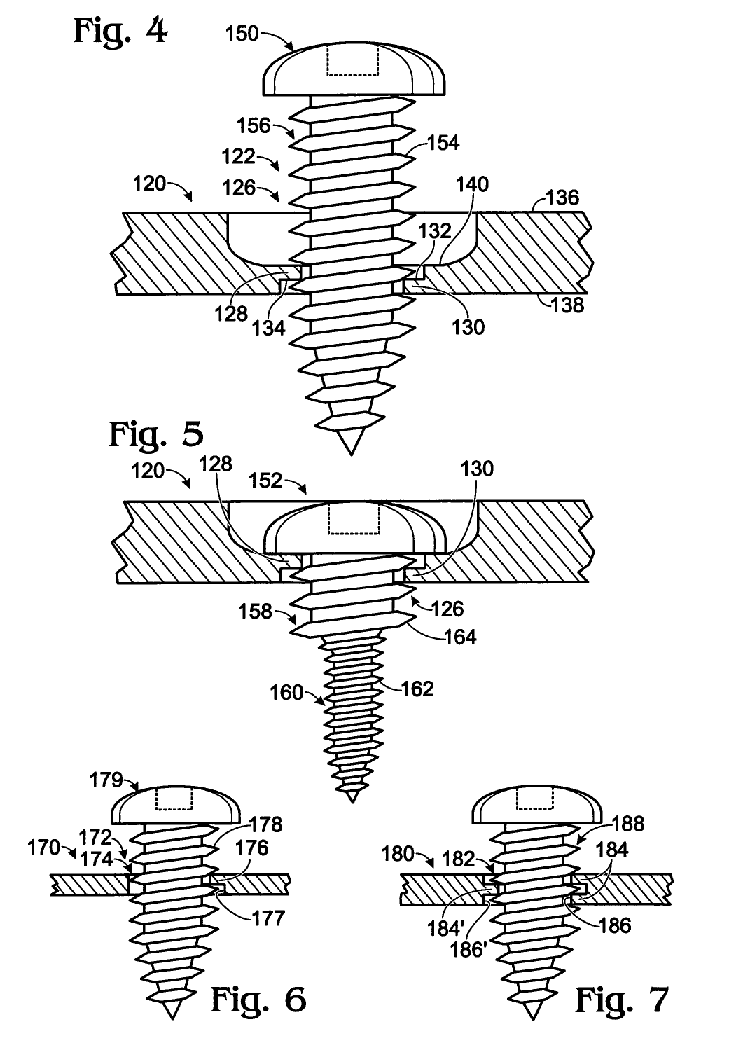

FIGS. 3 and 4 show plan and sectional views, respectively, of a portion of a bone plate 120 including one or more combination apertures 122. Combination aperture 122 may include one or more nonlocking regions 124 and one or more locking regions 126 in any suitable relative arrangement. For example, these regions may alternate or repeat, as desired. These regions may be disposed along a line and/or arc, may be offset from one another, and/or may define an angle, among others. Furthermore, these regions may adjoin one another or may be disposed in a spaced relation within the aperture. The combination aperture may extend in a direction that is parallel, oblique, or transverse to a long axis of the bone plate.

Nonlocking and locking regions 124, 126 may have any suitable structure. Nonlocking region 124 may lack retention structures that engage and retain a thread of a fastener. Nonlocking region 124 may include a circular opening, as shown in FIG. 3, or may include an elongate opening, such as an oval or arcuate opening, among others. The nonlocking region may have a width great enough to permit passage of a threaded shank of a fastener without engagement of the shank with the aperture, but small enough to restrict passage of the head of the fastener. Locking region 126 may be elongate, as shown in FIG. 3, or may be circular, among others.

The locking region may include retention structures 128, 130, such as ridges extending along the long axis of the nonlocking region of the combination aperture. Retention structures 128, 130 may be longitudinally offset from one another, for example, by opposing recesses or detents 132, 134 formed on opposing outer and inner surfaces 136, 138, respectively, of the plate. The recesses may be defined by recessed surfaces on opposing faces of the bone plate. The recessed surfaces may be at least substantially parallel to the outer and/or inner surfaces of the bone plate, or may extend obliquely relative to these surfaces. In some examples, one or both of these recessed surfaces may smoothly transition to their respective, adjacent outer or inner surfaces (or to a counterbore surface). In some examples, the locking region may include a helical thread, or one or more segments thereof, disposed, for example, in the circular end region of the aperture, in addition to or instead of the retention structures.

The combination aperture may include at least one counterbore 140. The counterbore may extend at least substantially around the combination aperture, as shown in FIGS. 3 and 4, or may extend around only a portion of the combination aperture. In some examples, the aperture may include a plurality of distinct counterbores disposed adjacent the nonlocking region 124 and the locking region 126. The distinct counterbores may be contiguous or spaced.

FIGS. 4 and 5 show two distinct bone screws 150, 152 that may be placed in locking region 126 of the combination aperture. Bone screw 150 depicted in FIG. 4 may include a thread 154 having an at least substantially constant pitch along threaded shank 156. Accordingly, this bone screw may be received by retention structures 128, 130, to advance through the aperture and into bone at the same rate. In contrast, bone screw 152 depicted in FIG. 5 may include proximal and distal threaded regions 158, 160, having a different diameter and/or a different pitch. In the present illustration, distal threaded region 160 has a thread 162 with a major diameter that is less than the lateral spacing of retention structures 128, 130. As a result, the distal threaded region can be passed through the locking region 126 without being engaged by the retention structures. On the other hand, proximal threaded region 158 may have a thread 164 with major and minor diameters that permit this threaded region to be received in the aperture rotationally and to be engaged by retention structures 128, 130 of the aperture. Threads 162, 164 may have the same or different pitches. For example, thread 162 may have a greater pitch than thread 164, so that the bone plate is compressed against the bone as thread 164 advances through the locking aperture. Alternatively, these threads may have the same pitch to permit the same rate of advancement of the bone screw relative to bone and the bone plate, so as to avoid compression of the bone by the bone plate and possibly to preserve a desired gap between the plate and the bone.

FIG. 6 shows a sectional view of a portion of a bone plate 170 including an aperture 172 that may be a locking aperture or a combination locking/nonlocking aperture. In this example, a locking region 174 of aperture 172 is similar to locking region 126 of FIGS. 4 and 5, except that locking region 174 includes only one retention structure 176 disposed on one opposing side of the aperture, defined by a recess or detent 177 formed on the inner or bone-facing surface of plate 170. A second opposing side of the aperture does not include any retention structures, and is shaped to form a surface oriented at least substantially orthogonal to both the bone-facing and bone-opposing surfaces of the plate along the entire thickness of the aperture. A thread 178 of a fastener 179 is configured to enter the aperture and to engage the retention structure.

Example 2

### Bone Plates with Longitudinally Arrayed Ridges

This example describes, without limitation, exemplary bone plates having longitudinally arrayed ridges; see FIG. 7.

Bone plate 180 of FIG. 7 may include a locking aperture 182 having two or more ledges or ridges 184, 184′ arrayed longitudinally on one more walls 186, 186′ of the aperture. In the present illustration, wall 186 has two ridges 184, and opposing wall 186′ has one ridge 184′. However, each wall may have any suitable number of ridges, including no ridges. The number of ridges may be selected, for example, according to the thickness of the plate, the pitch of a fastener to be placed in the aperture, and/or according to manufacturing constraints, among others. With two ridges arrayed longitudinally On a wall, the ridges may have a spacing that is approximately equal to the pitch of a thread on a threaded shank 188 to be used in conjunction with the locking aperture. As FIG. 7 indicates, one of the two or more ridges arrayed longitudinally on the same portion of the wall may be disposed at least substantially flush with a bone-facing surface of the bone plate, and another of the ridges may be disposed at least substantially flush with a bone-opposing surface of the bone plate, with the two ridges separated by an intermediate recess.

### Example 3

### Exemplary Configurations of Apertures and Fasteners

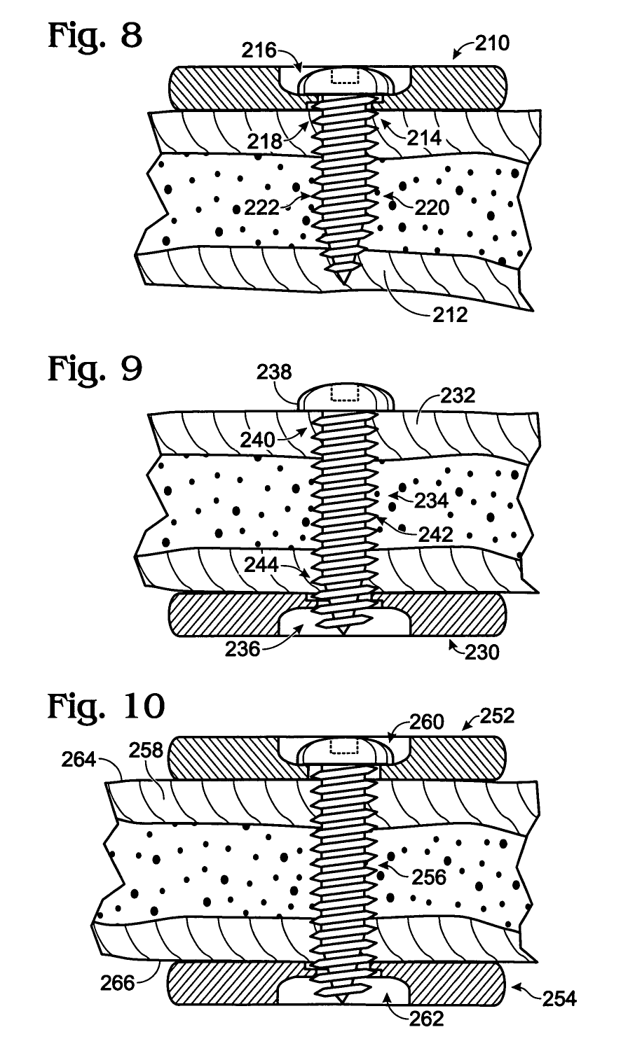

This example describes, without limitation, exemplary configurations in which locking apertures may receive fasteners; see FIGS. 8-10.

FIG. 8 shows a first bone plate 210 secured to a bone 212 with a bone screw 214 that is received in a locking aperture 216 of the bone plate. Here, a proximal region 218 of a threaded shank 220 of the bone screw may be engaged with the locking aperture of the bone plate, and a distal region 222 of the threaded shank may be spaced from the bone plate and engaged with the bone.

FIG. 9 shows a second bone plate 230 secured to a bone 232 with a bone screw 234 that is received in a locking aperture 236 of the bone plate. Here, a head 238 and a proximal region 240 of a threaded shank 242 of the bone screw may be spaced from the bone plate, and a distal region 244 of the threaded shank may be engaged with the locking aperture of the bone plate.

FIG. 10 shows third and fourth bone plates 252, 254 (or plate portions) spanned by a bone screw 256 and a bone 258. Here, the bone screw may be placed through a first aperture 260 of the first plate or plate portion 252, and then received distally by a second aperture 262 disposed in the second plate or plate portion 264. First aperture 260 and/or second aperture 262 may be a locking aperture. In an exemplary configuration, first aperture 260 is a slot that is nonlocking, and second aperture 262 is a slot that is locking (or a combination nonlocking/locking aperture; see Example 1). In some examples, the plate portions may be formed integrally in one plate, such as a U-shaped plate configured to extend between opposing surfaces 264, 266 of bone 258, such as a rib or clavicle bone. Accordingly, the bone plate may extend transversely on the bone. The opposing surfaces may be have relative positions that are anterior and posterior, superior and inferior, dorsal and ventral, proximal and distal, medial and lateral, and/or the like. Further aspects of bone plates for use on a rib and/or a clavicle bone and that may include one or more locking apertures are described in the following patent applications, which are incorporated herein by reference in their entirety for all purposes: U.S. Provisional Patent Application Ser. No. 60/548,685, filed Feb. 26, 2004; and U.S. patent application Ser. No. 10/927,824, filed Aug. 27, 2004.

### Example 4

### Selected Embodiments

This example further describes selected aspects and embodiments of the present teachings, presented as a series of numbered paragraphs.

1. A bone plate for fixing a bone, comprising a body configured to engage and support a bone and including a wall defining an elongate aperture, the elongate aperture defining a long axis, wherein the wall includes retention structures formed by opposing portions of the wall and offset longitudinally from one another so that the retention structures can retain a fastener advanced rotationally into the aperture by engagement with a threaded shank of the fastener.

2. The bone plate of paragraph 1, the body including a bone-facing surface and a bone-opposing surface, wherein one of the retention structures is spaced from the bone-facing surface by a recess in the bone-facing surface, and wherein another of the retention structures is spaced from the bone-opposing surface by a recess in the bone-opposing surface.

3. The bone plate of any preceding paragraph, the retention structures being ridges, the aperture having a first width measured between the ridges, wherein the wall also defines an opening adjoining the aperture, and wherein the opening has a second width greater than the first width, so that the opening can receive the threaded fastener without retaining the threaded fastener.

4. The bone plate of paragraph 3, wherein the opening is defined by a circular portion of the wall, and wherein the ridges do not extend substantially into the circular portion of the wall.

5. The bone plate of any preceding paragraph, wherein the wail also defines a counterbore configured to at least partially receive a head of the threaded fastener, and wherein the counterbore at least substantially extends around the aperture.

6. The bone plate of any preceding paragraph, wherein the body defines an opening that is aligned with the aperture and spaced therefrom, and wherein the opening is configured to permit the threaded fastener to extend through the opening to be received and retained in the aperture.

7. The bone plate of paragraph 6, wherein the opening is also elongate so that the threaded fastener can be placed through the opening at a plurality of distinct positions along the opening to be received at corresponding aligned positions of the elongate aperture.

8. The bone plate of any preceding paragraph, wherein the bone plate includes a U-shaped portion having opposed surfaces configured to face opposite sides of a bone.

9. The bone plate of any preceding paragraph, the aperture defining a length-by-width plane, wherein the retention structures extend at least substantially parallel to the plane.

10. The bone plate of any preceding paragraph, wherein the retention structures are at least generally linear along a substantial portion of their lengths.

11. The bone plate of any preceding paragraph, wherein the retention structures are arcuate and nonhelical along a substantial portion of their lengths.

12. The bone plate of any preceding claim, the body including a bone-facing surface and a bone-opposing surface, wherein a first of the retention structures is a first ledge disposed substantially flush with the bone-facing surface, wherein a second of the retention structures is a second ledge disposed substantially flush with the bone-opposing surface and separated from the first ledge by an intermediate recess, and wherein a third of the retention structures is a third ledge spaced from the bone-facing surface by a first recess and spaced from the bone-opposing surface by a second recess.

13. A bone plate for fixing a bone, comprising a body configured to engage and support a bone and including a wall defining an elongate aperture, the elongate aperture defining a plane and a long axis, wherein the wall includes retention structures formed by opposing portions of the wall and offset from one another along the vertical axis so that the retention structures can retain a fastener advanced rotationally into the aperture by engagement with a threaded shank of the fastener.

14. The bone plate of any preceding paragraph, wherein the thread spacing and/or plate offset are selected from the values in Table 1.

15. The bone plate of any preceding paragraph, wherein the body further includes at least one nonlocking aperture.

16. A bone plate for fixing a bone, comprising (A) a body configured to engage and support a bone; (B) a wall in the body defining an aperture having first and second substantially opposing sides defining a thickness of the aperture and configured to receive a threaded fastener for fastening the bone plate to the bone; and (C) a retention structure formed by the wall and configured to rotationally engage the fastener when the fastener is inserted into the aperture and contacts the retention structure; wherein the retention structure is a ledge disposed on the first opposing side of the aperture, substantially flush with a bone-opposing surface of the body and separated from a bone-facing surface of the body by a recess; and wherein the second opposing side of the aperture is shaped to define a surface oriented substantially orthogonal to both the bone-facing surface and the bone-opposing surface along the entire thickness of the aperture.

17. A system or kit for fixing a bone, comprising (A) a bone screw; and (B) a bone plate according to any of paragraphs 1-16.

18. The system or kit of paragraph 17, wherein the bone screw includes a thread with a pitch measured between adjacent segments of the thread, and wherein the retention structures are offset by a distance of approximately one-half the pitch.

19. The system or kit of paragraph 17 or 18, wherein the bone screw includes a threaded shank having a major diameter and a minor diameter, and wherein the retention structures are spaced, as measured parallel to the plane of the aperture, by a distance less than the major diameter.

20. The system or kit of paragraph 19, wherein the retention structures are spaced by greater than the minor diameter.

21. the system or kit of paragraph 19, wherein the aperture includes an opening having a width greater than the major diameter of the threaded shank, so that the bone screw can be placed into the opening without being retained therein.

22. A method of fixing a bone, comprising (A) selecting a bone plate having at least one aperture with longitudinally offset retention structures; (B) selecting a fastener having a threaded shank; and (C) securing the fastener to the bone and the aperture by rotating a threaded shank of the fastener into the bone and the aperture, so that the fastener is locked to the bone plate by engagement of the threaded shank with the retention structures.

23. A method of fixing a bone, comprising (A) selecting a bone plate according to any of paragraphs 1-16 or a system or kit according to any of paragraphs 17-21; and (B) securing the bone plate to the bone, at least partially, by placing the bone screw into the bone and in the aperture to be retained therein by the retention structures.

24. The method of paragraph 23, wherein the fastener is placed in the aperture before the bone.

25. The method of paragraph 23, wherein the fastener is placed in the bone before the aperture.

26. A method of fixing a bone, comprising (A) selecting a bone plate according to paragraph 3; (B) selecting one of the opening and the aperture; and (C) placing a threaded fastener into the bone and the selected one of the opening or aperture, the fastener to be engaged by the retention structures.

27. A method of fixing a bone, comprising (A) selecting a bone plate according to paragraph 6; (B) securing the bone plate at least partially to the bone by placing a threaded fastener through the opening, into the bone, and into engagement with the retention structures so that the threaded fastener is locked to the bone plate.

28. A method of fixing a bone, comprising (A) selecting a bone plate having an aperture with at least one retention structure; (B) selecting a fastener having a threaded shank, the threaded shank having a thread that corresponds in pitch to the retention structure; and (C) advancing the threaded shank rotationally into the bone and into engagement with the retention structure of the aperture, wherein further advancing rotationally of the threaded shank maintains the bone plate at an at least substantially constant spacing from the bone.

The disclosure set forth herein may encompass one or more distinct inventions, each with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present teachings.

1. A bone plate for fixing a bone, comprising a body configured to engage and support a bone and including a wall defining an elongate aperture, wherein the wall includes retention structures formed by opposing portions of the wall and offset longitudinally from one another so that the retention structures can retain a fastener advanced rotationally into the aperture by engagement with a threaded shank of the fastener.

2. The bone plate of claim 1, the body including a bone-facing surface and a bone-opposing surface, wherein one of the retention structures is spaced from the bone-facing surface by a recess in the bone-facing surface, and wherein another of the retention structures is spaced from the bone-opposing surface by a recess in the bone-opposing surface.

3. The bone plate of claim 1, the retention structures being disposed at least partially in a first region of the aperture, wherein the aperture further includes a second region adjoining the first, the second region configured to allow passage of a threaded shank of the fastener without the retention structures engaging the threaded shank.

4. The bone plate of claim 3, wherein the first region is at least substantially oval in shape and configured to allow selective placement of the fastener at a plurality of positions within the first region, wherein the second region is at least substantially circular in shape, and wherein the retention structures do not extend substantially into the second region.

5. The bone plate of claim 1, wherein the wall further defines a counterbore extending at least substantially around the aperture and configured to at least partially receive a head of the threaded fastener.

6. The bone plate of claim 1, the aperture defining a length-by-width plane, wherein the retention structures extend at least substantially parallel to the plane.

7. The bone plate of claim 1, wherein the retention structures are at least generally linear along a substantial portion of their lengths.

8. The bone plate of claim 1, wherein the retention structures are offset longitudinally by an offset distance correlated to a thread pitch of the threaded fastener.

9. The bone plate of claim 8, wherein the offset distance is approximately one-half the pitch.

10. The bone plate of claim 9, wherein the body adjacent the aperture has a thickness approximately equal to the pitch.

11. The bone plate of claim 1, wherein the body further defines at least one nonlocking aperture.

12. A kit for fixing a bone, comprising:
a bone screw; and
a bone plate according to claim 1.

13. The kit of claim 12, the bone screw including a thread with a pitch measured between adjacent segments of the thread, wherein the retention structures are offset by a distance of approximately one-half the pitch.

14. The kit of claim 12, the bone screw including a threaded shank having a major diameter and a minor diameter, wherein the retention structures are spaced, as measured parallel to the plane of the aperture, by a distance less than the major diameter.

15. The system or kit of claim 14, wherein the retention structures are spaced by a distance greater than the minor diameter.

16. The kit of claim 14, Wherein a portion of the aperture has a width greater than the major diameter of the threaded shank, so that the bone screw can be placed into the opening without being retained therein.

17. A method of fixing a bone, comprising:
selecting a bone plate according to claim 1; and
securing the bone plate to the bone, at least partially, by placing the fastener into the bone and in the aperture to be retained therein by the retention structures.

18. The method of claim 17, wherein the fastener is placed in the aperture before the bone.

19. The method of claim 17, wherein the fastener is placed in the bone before the aperture.
